(19) United States
(12) Patent Application Publication
(10) Pub. No.: US 2009/0173353 A1
(43) Pub. Date: Jul. 9, 2009

(54) Self-fitting device for location in an ear canal

(75) Inventors: Ricky Wayne Purcell, Alpharetta, GA (US); Debra N. Welchel, Woodstock, GA (US); Robert A. Drew, Atlanta, GA (US); Shawn E. Jenkins, Duluth, GA (US); Jeffrey Jennings Krueger, Roswell, GA (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 12/002,021

(22) Filed: Dec. 14, 2007

(51) Int. Cl. A61F 11/10; A61F 11/00

(52) U.S. Cl. 128/865; 181/135

(57) ABSTRACT

Self-fitting device for location in ear canal and related method, including a bladder joined with a pump assembly via a valve assembly. The pump assembly transfers air to the bladder and achieves a predetermined pressure with the chamber regardless of ear canal size. The predetermined pressure may be achieved by reducing excess air pressure through the valve assembly.

### BACKGROUND OF THE INVENTION

The present invention relates generally to devices for location in an ear canal, and more particularly to such devices being self-fitting and used for sound optimization such as noise reduction and/or acoustic enhancement.

The need for adequate hearing protection in high noise environments has long been recognized among those concerned with health and safety issues, and much effort has gone into providing such protection. However, most experts in this field would acknowledge that this effort has not been very successful. Protective devices have proliferated yet remained mediocre in performance. Workers in high noise environments who should use these devices often do not, or use them only under duress from their employers. Individuals that work in high noise environments rarely understand that the effects of high noise exposure are not limited to the moment but are cumulative as well. The lack of worker compliance with safety rules is exacerbated by the fact that currently available hearing protection devices are often uncomfortable, clumsy to use, and/or perform poorly. Fortunately, as hearing protection devices become more comfortable and perform better, worker compliance with their use should also improve.

For example, existing disposable foam ear plugs are uncomfortable for wearers with small ear canals, and are difficult to properly insert. Common disposable foam ear plugs require the user to compress the area of the plug and insert it into the ear canal where it then attempts to re-expand. This method can cause discomfort for people with small ear canals in that the more compressed the ear plug, the greater its exerted force toward re-expansion. Further, existing disposable foam ear plugs require the user to roll the foam between their fingers to compress the foam to a sufficient size for proper insertion. If this step is not done, or is insufficiently done, the ear plug is often inserted improperly so as to not provide optimal protection. Also, if the user has dirty hands when compressing the ear plug, dirt and/or germs are then put into the ear canal with the inserted ear plug. Furthermore, existing foam ear plugs are often designed for one time use.

Accordingly, while various types of in-ear devices exist in the art, there remains a need for an in-ear device that helps overcome one or more of the aforementioned problems.

### SUMMARY OF THE INVENTION

Various definitions used throughout the specification and claims are provided first, followed by a description of various aspects of the invention. One aspect of the present invention is a self-fitting device for location in an ear canal. The device includes a body having a body ear end and an opposite body user end. The body user end includes a pump assembly and a valve assembly. The body ear end includes a bladder defining a chamber, wherein the chamber is in communication with the pump assembly via the valve assembly. The device has an activated state and an inactivated state. The valve assembly is adapted to release air from the body ear end to the ambient surroundings at a predetermined pressure to maintain the chamber at the predetermined pressure when the device is in the activated state.

In another aspect of the invention, there is a method for self-fitting the device to an ear canal, the device having an inactivated state and an activated state. The method steps include providing the device, the device comprising a body having a body ear end and an opposite body user end, wherein the body ear end defines a chamber; locating the body ear end in the ear canal in the inactivated state; and activating the device to the activated state by transferring a fixed volume of air to the body user end; and pressurizing the chamber by releasing any excess air to an ambient surrounding to achieve a predetermined pressure within the chamber.

In yet another aspect of the invention, there is another method for self-fitting device to an ear canal, the device having an activated state and an inactivated state. The method includes the step of providing the device, the device comprising: a pump assembly adapted to contain air; a valve assembly having a first end and a second end; and a bladder that defines a chamber adapted to contain air: wherein the bladder is attached to the first end of the valve assembly, and wherein the pump assembly is attached to the second end of the valve assembly. The valve assembly includes a central valve for receiving air from the pump assembly, and a side valve that emits air from the valve assembly to ambient surroundings at a predetermined pressure. Further method steps include: locating the bladder in the ear canal when the device is in the inactivated state; activating the device to the activated state by transferring air from the pump assembly to the chamber, and pressurizing the chamber to the predetermined pressure.

As used herein, “resilient” means that property of a material or composite material that permits it to be deformed in size and/or shape and then recover at least about 80% of its original size and shape no later than two minutes after removal of the force causing the deformation.

As used herein, “non-resilient” means the opposite of resilient.

As used herein, the terms “integral” and “integrally” refer to a non-discrete portion of an object. An integrally formed portion of an object can differ from one that is coupled to the object, since the integrally formed portion of the object typically does not form an interface with a remaining portion of the object.

As used herein, “connected” means the joining, adhering, bonding, attaching, or the like, of two elements. Two elements will be considered to be connected together when they are connected directly to one another or indirectly to one another, such as when each is directly connected to intermediate elements.

As used herein, the term “valve” means any device that controls, arrests, or releases the flow of air. Each of the valves of the present invention are combination valves, meaning that in one flow direction they operate as a relief valve that is triggered at a predetermined pressure, and in the opposite flow direction they operate as a free-flowing valve.

Still other features of the invention will be in part apparent and in part pointed out hereinafter as well as better understood by practice of the invention. It is to be understood that both the foregoing general description and the following detailed description are exemplary and are intended to provide further explanation of the invention claimed. The accompanying drawings, which are incorporated in and constitute part of this specification, are included to illustrate and provide a further understanding of the self-fitting device for location in an ear canal that is the present invention.

### BRIEF DESCRIPTION OF THE DRAWINGS

FIG. 1 is a side cross-sectional view of the self-fitting device for an ear canal of the present invention in an at-rest position;

FIG. 2 is a side cross-sectional view of the device in FIG. 1, but now in an in-ear position;

FIG. 2A is a partial view of the device of FIG. 1, showing schematically an embodiment of the valve assembly with air flowing in a direction related to activation of the pump assembly;

FIG. 2B is a partial I view of the device of FIG. 1, showing a side perspective view of the valve assembly;

FIG. 2C is another view of the valve assembly shown in FIG. 2A, with the air flowing in an opposite direction, related to the deactivation of the pump assembly;

FIG. 3 is a is perspective view of the device in FIG. 2;

FIG. 4 is a perspective view of the device in FIG. 2, as after insertion and after a user has made ready;

FIG. 4A is a partial front view of the frame portion and pump bladder of the device shown in FIG. 1; and

FIG. 4B is a bottom view of the frame shown in FIG. 4A.

Corresponding reference characters indicate corresponding parts throughout the drawings.

### DETAILED DESCRIPTION

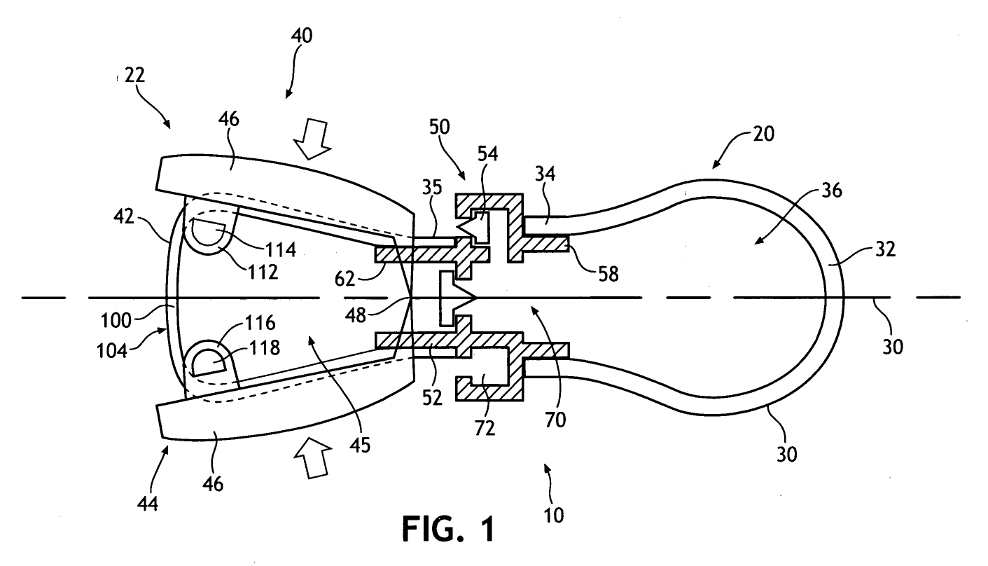

Referring now to the drawings, and in particular FIGS. 1-3, there is depicted a self-fitting device 10 for location in an ear canal 12. The device 10 has a body ear end 20, and a body user end 22. In particular, in FIG. 3, shown is a device 10 inserted into an outer ear 13 which is joined to the ear canal (not seen). Body ear end 20 includes a bladder 30, connected to the body user end 22 which includes a pump assembly 40. The body user end further includes a valve assembly 50, which serves to connect bladder 30 to pump assembly 40. The valve assembly 50 includes a central valve 52 and a side valve 54 which together, facilitate a predetermined pressure within the bladder 30 after the device 10 has been inserted into an ear canal and subsequently activated.

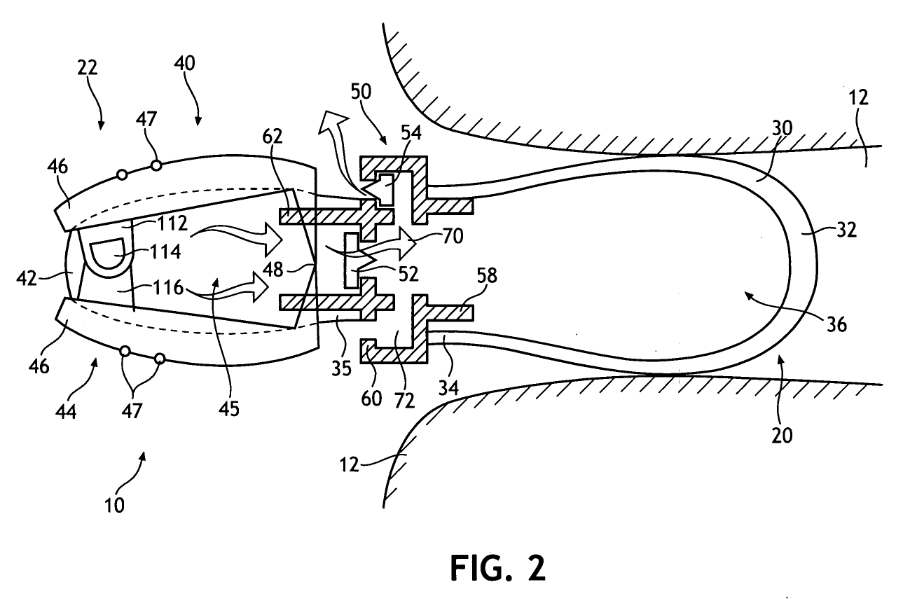

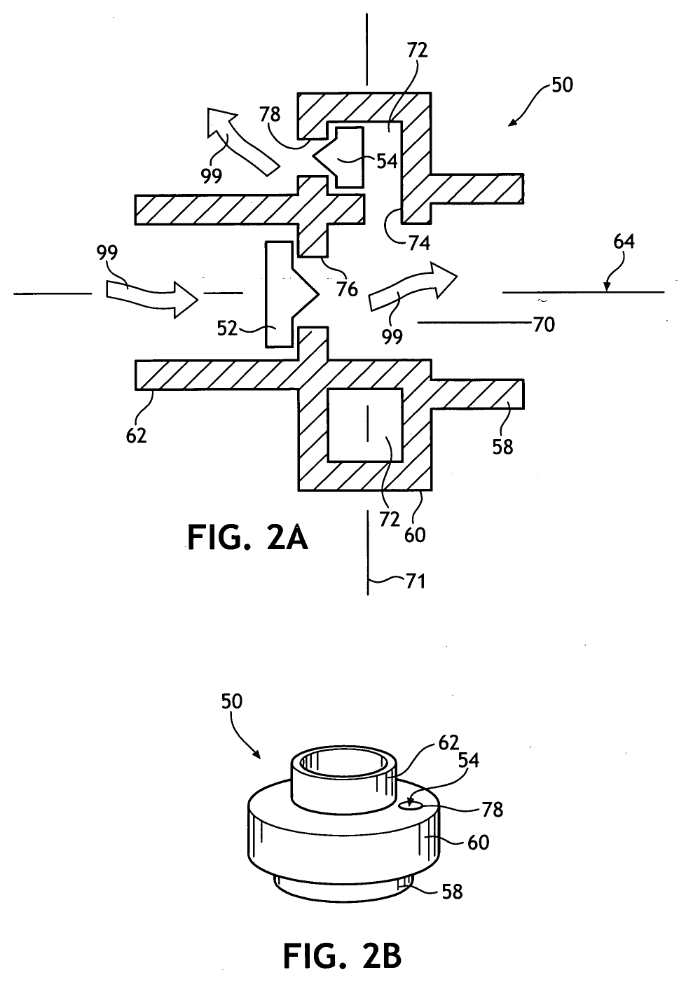

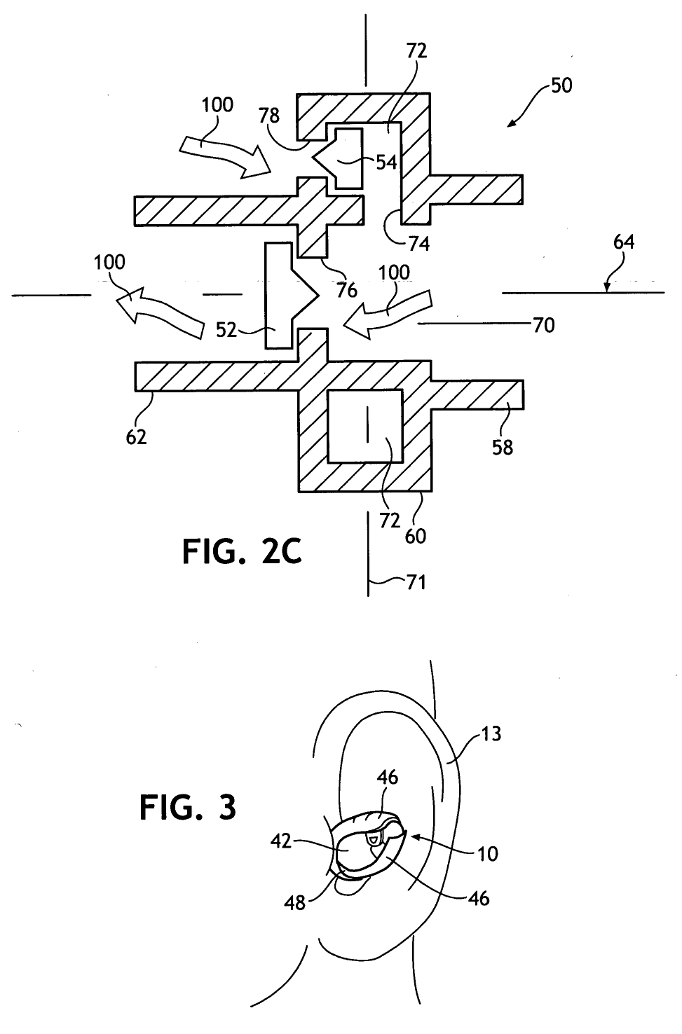

Desirably, the bladder 30 is resilient. Bladder 30 includes two main wall portions, a main body 32 and a collar portion 34 that are desirably, integrally connected together. For example, these two parts could be formed together of one material, or formed of separate materials that are sequentially formed together, or formed separately and then joined together by any conventional means (e.g., adhesive, chemical or heat or other similarly resulting mechanical bonded relationship). A chamber 36 (seen best in FIGS. 1-2) is defined by the bladder wall portions. Chamber 36 is in fluid communication with pump 40 via the valve assembly 50.

The bladder 30 is deformable between an insertion position (FIG. 1) and an in-ear position (FIG. 2). It is noted that the shapes of the bladder 30 seen in FIGS. 1-2 are illustrative of the relative differences between insertion, and in-ear positions, but such shapes are not so limited (e.g., the bladder in FIGS. 1 and 2 may be more or less bulbous). The shape of the bladder 30 when in the in-ear position is determined in part by the shape of the ear canal 12.

Referring now to FIGS. 2A and 2B, the valve assembly 50 defines three sections; a bladder neck 58, a valve chamber 60, and a pump neck 62. The bladder neck 58, valve chamber 60, and pump neck 62 are in series, and are (1) symmetrically situated about a longitudinal axis 64, and (2) may also be concentrically configured about longitudinal axis 64. The bladder neck 58 and pump neck 62 may be cylindrical, and may be integrally connected to opposite ends of the valve chamber 60. For example, these two parts could be formed together of one material, or formed of separate materials that are sequentially formed together, or formed separately and then joined together by any conventional means (e.g., adhesive, chemical or heat or other similarly resulting mechanical bonded relationship).

Valve chamber 60 may have an inner chamber 70 in fluid communication with an outer chamber 72 via a channel 74. Two other channels allow air to pass through the valve chamber 60, namely an inner channel 76 and an outer channel 78. The central valve 52 is operatively connected to the inner channel 76, and the side valve 54 is operatively connected to the outer channel 78.

Central valve 52 and side valve 54 allow a two-way passage of air under qualifying conditions. Generally, each valve 52, 54 operates as a relief valve at a predetermined pressure in one direction, and as a free-flowing valve in the opposite direction. Referring to FIG. 2A, air flow through the valve assembly 50 is depicted by reference arrows 99. During activation of the pump assembly, the pressure inside the bladder 42 increases and the central valve 52 allows air to flow into bladder 30 once the pressure within bladder 42 is equal to or greater than the central-valve predetermined pressure. Next, the pressure inside the bladder 30 increases and the side valve 54 allows air to flow to the ambient surroundings of device 10 once the pressure within bladder 30 is equal to or greater than the side-valve predetermined pressure. Referring now to FIG. 2C, air flow through the valve assembly 50 is depicted by reference arrows 100. During deactivation of the pump assembly, the resulting negative pressure inside bladder 42 and bladder 30 forms a vacuum. When a vacuum occurs, the side valve 54 allows air to freely flow back into the valve assembly 50. A volume of air is pulled back into the pump assembly bladder 42 freely through central valve 52 so that it may return substantially to its original shape.

Valve chamber 60 may be constructed from a plastic or metal material. In one non limiting example, valve chamber 60 is constructed from plastic, and is pieced together from two disparate parts joined permanently at about the axis 71 (see FIG. 2A) by welding, adhesive, or the like.

The pump assembly 40 includes two parts, a pump bladder 42 and a frame 44. Similar to bladder 30, the pump bladder 42 may include two main wall portions, a pump body 100 and a collar portion 35 that are desirably, integrally connected together. For example, these two parts could be formed together of one material, or formed of separate materials that are sequentially formed together, or formed separately and then joined together by any conventional means (e.g., adhesive, chemical or heat or other similarly resulting mechanical bonded relationship). A chamber 45 (seen best in FIGS. 1-2) is defined by the pump bladder wall portions. Chamber 45 is in fluid communication with chamber 36 via the valve assembly 50.

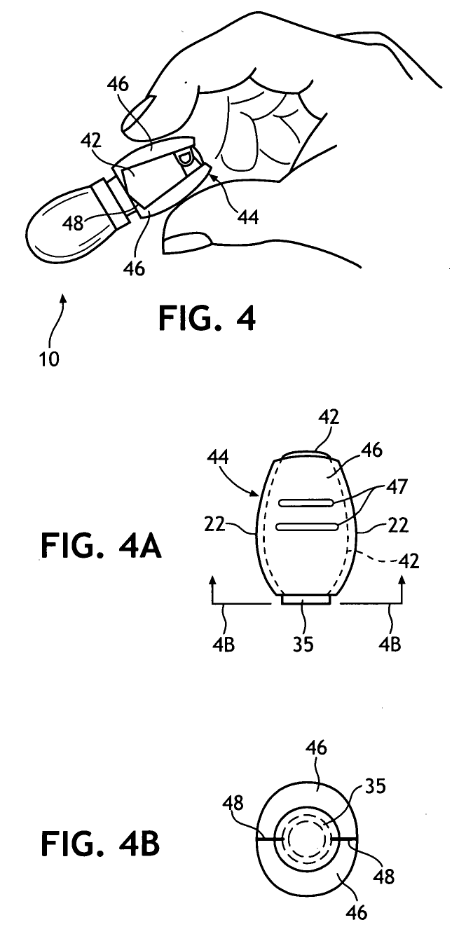

The pump bladder 42 is deformable between an inactivated position (FIG. 1) to an activated position (FIG. 2). It is noted that the shapes of the bladder 42 seen in FIGS. 1-2 are illustrative of the relative differences between when bladder 30 is pressurized and in the ear, and non-pressurized. Desirably, the shape of the pump bladder 42 when in the inactivated position is such that it the wall portions converge from a distal end 104 to the collar portion 34. Thus, the distance between the longitudinal axis 64 and the pump wall portions increases from the collar portion 34 to the distal end 104. The side resulting cross-sectional view of pump bladder 42 is desirably a wedge shape so that a greater volume of air may be transferred from the bladder 42 to the bladder 30 with a single pump motion as described below. When the pump bladder 42 is in the activated position, its shape is largely determined by the frame 44. However, as the bladder 42 is compressed by squeezing arms 46 together, it is desirable that the bladder 42 not expand beyond the perimeter 22 (FIG. 4A) defining each arm 46 to prevent the bladder 42 from putting undesirable force on the outer ear 13. The activation of the pump assembly may provide for the expulsion of a predetermined and repeatable amount of air from bladder 42 to bladder 30. Desirably, bladder 42 holds a substantially fixed volume of air in an inactivated state for the purpose of repeatability. The much desired resilient quality of bladder 42 allows it to return substantially to its original shape, and thus, hold the fixed volume within.

Referring to FIGS. 3, 4, and 4A, the frame 44 has a pair of arms 46 connected together at a hinge 48. The frame arms 46 may be cupped such that they make direct contact with the outer surface of bladder 42. Such direct contact facilitates the operation of frame 44, the purpose of which is to transfer air from the interior chamber 45 of bladder 42 to the chamber 36 of bladder 30. Thus, the more surface area contacted by each arm 46 after the activation of pump 40 is complete, the more efficient the air transfer. Each arm 46 may be symmetrical and similar or identical in overall shape. Desirably, the arms 46 may be constructed from plastic such as polyethylene or the like, and have an integral or non-integral connection at the hinge 48. For instance, the hinge 48 may be an integral flexible seam between the two arms 46 (see FIG. 4B) or be provided by a separate joint material such as tape or the like. In the alternative, the arms 46 may be made from a metal material such as spring steel and have an integral flexible seam at hinge 48 or added joint material to form a flexible seam, such as a tape or the like.

To provide a better grip of between the fingers as demonstrated in FIG. 4, the arms may include a friction feature such as indentations, a raised design, or the like. In one non-limiting example, the arms 46 include a pair of ridges 47 for the purpose of providing a better grip and adding aesthetic interest.

Arms 46 may include a selective locking mechanism 110 so that once the pump is in an activated position, it stays locked until the wearer of the device 10 decides to unlock arms 46. One possible embodiment of a locking mechanism may be seen in FIGS. 1 and 2. In FIG. 1, the frame 44 is in an unlocked position, and in FIG. 2, the frame 44 is in a locked position. The locking mechanism 110 has a male fin member 112 which includes a boss 114, and a female fin member 116 having an aperture 118 for receiving the boss 114. The fin members are attached to the arms such that when the arms are rotated about the hinge 48, the fins meet each other on different planes that provide a friction or slip fit. Each fin member is attached to the arms 46 such that when the frame 44 is in a locked position and the device 10 is seated in a wearer's ear, the fin members are visible and accessible (see FIG. 3). This allows the wearer to unlock the locking mechanism without first having to remove the device 10 from the ear, if desired. In operation, the arms 46 are squeezed together until the boss 114 fits into the aperture 118. There may be a second pair of fin members (not shown) symmetrically located on the opposite side of pump 40.

The device 10 is assembled by inserting the pump bladder 42 in between the arms 46 of frame 44 (see FIGS. 4A and 4B), and attaching the pump collar portion 35 to the pump neck 62. The bladder 30 is attached to the valve assembly 50 by attaching the bladder collar portion 34 to the bladder neck 58. The collar portions 34, 35 are effectively connected to bladder neck 58 and pump neck 62, respectively, such that when the device 10 is in an activated position, the pressure within chamber 36 remains elevated with respect to the inactivated position. An adhesive may be placed between the bladders 36 and 42 and the valve assembly 50 to maintain an effective, non-leaking connection.

While not required, it may be advantageous for sound enhancement, e.g., not only taking advantage of sound reduction capabilities but also audio or hearing-aid like capabilities. In this way, device 10 can be configured (not shown) to locate a speaker, microphone, or the like in device 10 and help bring desired sound into the ear canal and/or locate a speaker/microphone in the ear canal better, e.g., via valve assembly 40 or pump assembly 50 in communication with bladder 30.

The resilient bladders 30 and 42 may be made of a homogeneous material or a composite material, and may include one or more layers. Bladders 30 and 42 may be made of a polyurethane foam with a “skin” formed on the outside. It may be made from a flexible material that is injection or blow moldable into a bulb that does not permanently deform when stressed by squeezing. Examples could be polyurethane, santoprene, polyethylene, or polypropylene.

Bladder 30 may also have an additional flange (not shown) for enhanced sound reduction. For example, a flange may be located at the base of the bladder where it is in communication with the valve assembly 40, or at intermittent spacing along axis 64 in bladder main body 32.

Though not shown, a pair of devices 10, may be connected together by a lanyard or other similar connection means for a variety of reasons, e.g., easy location when not in an ear canal, to help remove from the ear canal, to keep from falling into a user's work space, or the like.

In other aspects of the invention there is provided an ear canal self-fitting device method. The method includes providing a device 10 having a pump assembly 40 located opposite a bladder 30. The method also includes disposing the bladder 30 into a wearer's ear canal such that the pump assembly is visible and accessible from the ear exterior 13. The method still further includes activating the pump assembly by squeezing the frame arms 46 together about the hinges 48. In addition to the step of activating the pump assembly 50, the arms 46 may be locked together with the locking mechanism 110. Activating the pump assembly causes the bladder 30 to be pressurized to a predetermined pressure. The predetermined pressure may be determined by sampling a population of potential wearers to obtain data regarding ear canal size, average ear canal pressure preferences, or other data relating to fit and comfort.

During use, should the device 10 experience a pressure decrease in chamber 36, the frame 44 may be unlocked and the pump assembly 40 reactivated. This will again raise the pressure inside chamber 36 to the predetermined pressure. Of course, if the pressure rises in chamber 36 during wear, the excess pressure will be automatically relieved by side valve 54. The wearer may selectively decide to deactivate device 10 to decrease sound protection. The device 10 may be removed from an ear with or without first deactivating the pump assembly.

In practice, device 10 may be used as follows. The device 10 would be located in ear canal 12 in an unexpanded, unactivated condition. A squeezing force can be applied to arms 46 (e.g., by a user's thumb and/or finger(s) or the like) causing air to transfer from the pump bladder 42 to the chamber 36 of the bladder 30. This air transfer will enlarge and/or reshape resilient bladder 30 to block the ear canal for sound optimization such as noise reduction and/or acoustic enhancement. Excess pressure is automatically relieved by the side valve 54. The in-ear position is determined by the user's particular ear canal shape and size and is therefore self-fitting or customizable. Comfort is optimized because regardless of the size of the wearer's ear canal, the bladder 30 exerts a uniform force upon the walls of the ear canal due to fact that the chamber 36 contains air at a predetermined pressure.

While not being required, it can be advantageous to that the following steps occur in the following sequential order: providing, followed by locating, followed by activating, followed by pressurizing.

In another aspect of the invention, there is another method for self-fitting device 10 to an ear canal 12, the device 10 having an activated state and an inactivated state. The method includes the step of providing the device 10, the device comprising: a pump assembly 40 adapted to contain air; a valve assembly 50 having a first end and a second end; and a bladder 30 that defines a chamber 36 adapted to contain air: wherein the bladder 30 is attached to the first end (neck 58) of the valve assembly, and wherein the pump assembly is attached to the second end (neck 62) of the valve assembly 50. The valve assembly 50 includes a central valve for receiving air from the pump assembly 40, and a side valve that emits air from the valve assembly 50 to ambient surroundings at a predetermined pressure. Further method steps include: locating the bladder 30 in the ear canal 12 when the device 10 is in the inactivated state; activating the device 10 to the activated state by transferring air from the pump assembly 40 to the chamber 36, and pressurizing the chamber 36 to the predetermined pressure.

The step of activating the device 10 may further include the steps of pivoting the pair of arms 46 about the hinge 48 to squeeze the pump bladder 42 and cause it to deform from an inactivated shape to an activated shape. Desirably, the arms 46 are selectively locked in the activated state while the device is being used in an ear canal. However, when desired, step of deactivating the device 10 may be achieved by pivoting the pair of arms 46 about the hinge 48 away from the pump bladder 42 to allow the pump bladder to substantially return to the inactivated shape.

Without being limited to a particular theory of understanding or noted advantageous features, the following features are discussed. The standardization of pressure within the chamber 36 reduces the diametrical force exerted on small ear canals, thereby enhancing comfort even for smaller ear canals. Also, with the features of the present invention it is made of sufficiently substantial materials and design so as to allow for multiple uses.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

1. A self-fitting device for location in an ear canal, comprising:
a body having a body ear end and an opposite body user end, the body user end comprising a pump assembly and a valve assembly, and;
the body ear end comprising a bladder defining a chamber, wherein the chamber is in communication with the pump assembly via the valve assembly,
wherein the device has an activated state and an inactivated state, and wherein the valve assembly is adapted to release air from the body ear end to the ambient surroundings at a predetermined pressure to maintain the chamber at the predetermined pressure when the device is in the activated state.

2. The device of claim 1 wherein the pump assembly comprises a pair of arms connected by a hinge.

3. The device of claim 2 wherein the pair of arms are integrally connected at the hinge.

4. The device of claim 2 wherein the pump assembly further comprises a pump bladder.

5. The device of claim 2 wherein the pump assembly further comprises a locking mechanism.

6. The device of claim 1 further comprising a microphone disposed in the valve assembly or pump assembly.

7. The device of claim 1 wherein the valve assembly comprises an inner channel with a central valve, and an outer channel with a side valve;
wherein the central valve and the side valve are adapted to allow air flow in two directions.

8. The device of claim 7 wherein side valve is in fluid communication with an ambient surrounding and the valve assembly.

9. The device of claim 7 wherein central valve is in fluid communication with the pump assembly and the valve assembly.

10. A method for self-fitting a device to an ear canal, the device having an inactivated state and an activated state, the method comprising the steps of:
providing the device, the device comprising a body having a body ear end and an opposite body user end, wherein the body ear end defines a chamber;
locating the body ear end in the ear canal in the inactivated state;
activating the device to the activated state by transferring a fixed volume of air to the body user end; and
pressurizing the chamber by releasing any excess air to an ambient surrounding to achieve a predetermined pressure within the chamber.

11. The method of claim 10 wherein the following steps occur in the following sequential order: providing, locating, activating, and pressurizing.

12. A method for self-fitting a device to an ear canal, the device having an activated state and an inactivated state, the method comprising the steps of:
providing the device, the device comprising:
a pump assembly adapted to contain air;
a valve assembly having a first end and a second end; and a
bladder that defines a chamber adapted to contain air:
wherein the bladder is attached to the first end of the valve assembly, and wherein the pump assembly is attached to the second end of the valve assembly; and
wherein the valve assembly comprises a central valve for receiving air from the pump assembly, and a side valve that emits air from the valve assembly to ambient surroundings at a predetermined pressure;
locating the bladder in the ear canal when the device is in the inactivated state;
activating the device to the activated state by transferring air from the pump assembly to the chamber, and
pressurizing the chamber to the predetermined pressure.

13. The method of claim 12 further comprising the step of relieving excess air from the chamber to the ambient surrounding through the side valve.

14. The method of claim 12 further comprising the step of locking the device in the activated state.

15. The method of claim 12 wherein the following steps occur in the following sequential order: providing, locating, activating, and pressurizing.

16. The method of claim 12 wherein the pump assembly comprises a frame surrounding a pump bladder, the frame comprising a pair of arms pivotally connected at a hinge, and
wherein the step of activating the device further comprises the steps of pivoting the pair of arms about the hinge to squeeze the pump bladder and cause it to deform from an inactivated shape to an activated shape.

17. The method of claim 16 further comprising the step of deactivating the device by pivoting the pair of arms about the hinge away from the pump bladder to allow the pump bladder to substantially return to the inactivated shape.

18. The method of claim 17 wherein the side valve emits air into the valve assembly from the ambient surroundings when the chamber is in a vacuum condition.

19. The method of claim 18 wherein the central valve emits air from the chamber to the pump assembly when the pump bladder is in a vacuum condition.

20. The method of claim 16 further comprising the step of locking the pair of arms together to maintain the activated state.
